(19) United States
(12) Patent Application Publication
(10) Pub. No.: US 2026/0234537 A1
(43) Pub. Date: Aug. 13, 2026

(54) CO-CULTURED CARDIOMYOCYTES ON CHIP (COCO CHIP) WITH PIEZOELECTRIC SENSING

(71) Applicant: THE BOARD OF TRUSTEES OF THE UNIVERSITY OF ARKANSAS, Little Rock, AR (US)

(72) Inventors: Kartik BALACHANDRAN, Fayetteville, AR (US); Alexis APPLEQUIST, Fayetteville, AR (US); Lance CORDES, College Station, TX (US)

(21) Appl. No.: 19/155,921

(22) PCT Filed: Feb. 12, 2024
(86) PCT No.: PCT/US2024/015366

§ 371 Date: Aug. 12, 2025

Related U.S. Application Data
(60) Provisional application No. 63/445,205, filed on Feb. 13, 2023.

(51) Int. Cl. C12M 1/34; C12M 1/00; C12M 1/32

(52) U.S. Cl. CPC C12M 41/46; C12M 23/12; C12M 23/20; C12M 23/34

(57) ABSTRACT

A cell-culture device for measuring forces exerted by cells includes a housing with one or more cell culture sections. Each cell culture section includes one or more compartments for seeding one or more cell types, and each compartment has a bottom surface that is coated with a layer of piezoelectric polydimethylsiloxane (PE-PDMS). Forces exerted by cells can be measured by seeding cells in the cell-culture device and detecting an electrical signal generated by the layer of PE-PDMS. The electrical signal is indicative of a mechanical movement converted to the electrical signal proportional to contractile force exerted from the cells. The electrical signal can be converted to an output of a contractile force measurement.

### CROSS REFERENCE TO RELATED APPLICATIONS

The present application is based on, claims priority to, and incorporates herein by reference in its entirety for all purposes, U.S. Provisional Application Ser. No. 63/445,205, filed Feb. 13, 2023.

### STATEMENT OF FEDERALLY SPONSORED RESEARCH

This invention was made with government support under W81XWH2010144 awarded by the Department of Defense. The government has certain rights in the invention.

### BACKGROUND

Organ-on-chip technology arose from the convergence of microfabrication and tissue engineering. Organ-on-chip systems can recreate complex organ functions, tissue-barrier properties, parenchymal tissue function, and multi-organ interactions. However, there are few benchtop technologies that can be used to study the physiological effects of new and standard pharmaceuticals on cardiomyocytes (either healthy or diseased) with non-invasive piezoelectric sensing.

### SUMMARY OF THE DISCLOSURE

The present disclosure addresses the aforementioned gap in the biomedical research field by providing a cell culture chip with co-culture capabilities and contractile force sensing piezoelectric materials.

In one non-limiting example of the present disclosure, the chip may be used to co-culture cardiomyocytes with other cell types. The addition of a piezoelectric measurement system and the option to test a plurality of cell types and/or drugs per chip has never been combined in a heart-on-chip design. Additionally, the design provides a numerical output to evaluate contractility, rather than the standard qualitative, visual examination of the cantilever activity used to calculate contractile strength.

In another aspect of the present disclosure, the chip may be further adapted to evaluate the contractile force generated by different cell types, such as skeletal muscle cells, smooth muscle cells, and neurons.

In one aspect of the disclosure, a cell-culture device for measuring forces exerted by cells is described, including: a housing with one or more cell culture sections, wherein each cell culture section includes one or more compartments for seeding one or more cell types, and each compartment has a bottom surface that is coated with a layer of piezoelectric polydimethylsiloxane (PE-PDMS).

In one aspect of the disclosure, a method for measuring forces exerted by cells is described, including: seeding cells in a cell-culture device as described above, where the layer of PE-PDMS on the bottom surface of the cell-culture device converts the mechanical energy of the contractile force to electrical energy and the summation of the electrical signal is output as a contractile force measurement.

In one aspect of the disclosure, a method of making a piezoelectric sensor is described, comprising: dissolving polyvinylidene fluoride-trifluoroethylene (P(VDF-TrFE)) in methyl-ethyl-ketone (MEK); mixing polydimethylsiloxane (PDMS) and curing agent at a ratio of 10:1 into the dissolved P(VDF-TrFE) to form piezoelectric PDMS (PE-PDMS); coating a surface of a substrate with a layer of the PE-PDMS to form the piezoelectric sensor; and positioning a base layer including one or more microelectrodes embedded thereon beneath the layer of PE-PDMS configured to detect a signal based on a deflection in the cantilevers.

### BRIEF DESCRIPTION OF THE DRAWINGS

FIG. 1A illustrates an exploded view of the cell-culture chip, according to aspects of the present disclosure.

FIG. 1B illustrates the exploded view of the cell-culture chip with the top glass slide inserted, according to the aspects of the present disclosure.

FIG. 1C illustrates an exploded view of the cell-culture chip, according to aspects of the present disclosure.

FIG. 1D illustrates the exploded view of the cell-culture chip with the top glass slide inserted, according to the aspects of the present disclosure.

FIG. 1E illustrates an example workflow for engineering a cell-culture chip and seeding it with cardiomyocyte cells, according to some aspects described in the present disclosure.

FIG. 1F is a schematic of printing gold electrodes on glass slides used in the cell-culture chips, according to aspects of the present disclosure.

FIG. 2 illustrates isometric view (panel A), front view (panel B), angled view (panel C) to show channels for shared media, and top-down view (panel D) of the 3D printed acrylonitrile butadiene styrene walls, according to the present disclosure.

FIG. 3A illustrates the cantilever placement on the completed chip with a top-down view, according to aspects of the present disclosure.

FIG. 3B illustrates the cantilever array which will be cut into the PE-PDMS micro-layer, according to aspects of the present disclosure.

FIG. 3C illustrates the cantilever structure and movement mechanism, according to aspects of the present disclosure.

FIG. 4 illustrates the electrode which can be used to provide electrical stimulation to the cardiomyocytes, according to aspects of the present disclosure.

FIG. 5 illustrates the contractile force measurement system, according to aspects of the present disclosure.

FIG. 6A illustrates a top view of the separate 2-part 3D printed molds.

FIG. 6B illustrates the orientation of the 2-part 3D printed molds relative to each other for creating PDMS walls, according to aspects of the present disclosure.

FIG. 6C illustrates a 3D printed support for drilling holes for fittings.

FIG. 6D illustrates an exploded view of the alternative chip design using molded walls, according to aspects of the patent disclosed.

FIG. 7 shows an example workflow according to aspects of the present disclosure.

FIG. 8 shows the voltage response of PE-PDMS material to manual substrate displacement shown in the red shaded sections.

FIG. 9 contains data of the varying percent PE-PDMS material and its respective stiffness.

FIG. 10 contains data of the varying percent PE-PDMS material and its respective sensitivity.

FIG. 11 is a plot of PE-PDMS sensitivity before and after subjection to fatigue testing.

FIG. 12A shows a plot of the mean height, max height, and thickness of 5% PE-PDMS films subjected to curing only, curing followed by annealing, and annealing followed by curing (n=4).

FIG. 12B shows plot of the mean height, max height, and thickness of 5% PE-PDMS films subjected to annealing for varying durations (0-90 min, n=4).

FIG. 13A is a cross sectional image of the uniformity of spin-coated thickness of PE-PDMS on glass coverslips.

FIG. 13B is a cross sectional image and corresponding thickness plot of the uniformity of spin-coated thickness of PE-PDMS on glass coverslips.

FIG. 13C is a plot of the spin-coating consistency of three samples of spin-coated PE-PDMS on glass coverslips.

FIG. 14 show fluorescence images of PE-PDMS autofluorescence. Scale bar=100 μm.

FIG. 15 shows phase/contrast images of pure PDMS (left) and PE-PDMS (right). Scale bar=100 μm.

FIG. 16 shows images of PE-PDMS before and after curing.

FIG. 17A is a plot of the sensitivity of an alternative piezo solution at different concentrations (n=3-4)

FIG. 17B is a plot comparing the sensitivities of PDMS with different piezo solutions at different concentrations (n=3-4)

FIG. 18 is a diagram of a sterile enclosure for continuous data collection from the cell-culture chip throughout cell culture.

FIG. 19A is a plot of the trace line of calcium concentration/calcium cycling.

FIG. 19B is a graph of beat frequency for human iPSC-derived cardiomyocytes culture on 0% and 5% PE-PEDMS (n=3).

FIG. 19C is a graph of peak Ca²⁺ concentration for human iPSC-derived cardiomyocytes culture on 0% and 5% PE-PEDMS (n=3).

FIG. 19D is a graph of Ca²⁺ decay for human iPSC-derived cardiomyocytes culture on 0% and 5% PE-PEDMS (n=3).

FIG. 20A is a plot of cell viability of cardiomyocytes cultured short-term (8 days) on 0% and 5% PE-PDMS.

FIG. 20B shows fluorescence images of cardiomyocytes cultured short term (8 days) on 0% and 5% PE-PDMS (scale bars=100 μm; DAPI (nucleus), TnT=cardiac troponin T, α-actinin, and phalloidin).

FIG. 21A is a plot of cell viability of cardiomyocytes cultured long-term (28 days) on 0% and 5% PE-PDMS coated substrates.

FIG. 21B shows fluorescence images of cardiomyocytes cultured long term (28 days) on 0% and 5% PE-PDMS (scale bars=100 μm).

FIG. 21C shows fluorescence image of cardiomyocytes cultured long-term (28 days) on 0% and 5% PE-PDMS (scale bars=100 μm).

### DETAILED DESCRIPTION OF THE DRAWINGS

The present disclosure presents a multi-chambered cell-culture platform with co-culture capabilities. The cell-culture platform may include, for example, an organ-on-a-chip device or other cell-culture chip device. In general, the cell-culture platform includes one or more individual culture sections, which may or may not be further divided into compartments for co-culturing two or more cell types. In one example, the platform may include nine individual culture sections which may contain two compartments per section for two or more cell types to be co-cultured with shared media. Solid walls may separate each of the nine sections. In a non-limiting example, human induced pluripotent stem cell (iPSC)-derived cardiomyocytes and human fibroblasts can be seeded in respective chambers to establish a co-culture environment. Alternatively, other cell types may be used, such as primary neonatal rat cardiomyocytes. In these instances, the cell-culture platform may be referred to as a heart-on-chip platform or device. Other cell types, such as endothelial cells, sympathetic neurons, mesenchymal stem cells, and epicardial cells may also be co-cultured with or without cardiomyocytes in the device.

The base of the heart-on-chip platform (i.e., where the cells are seeded onto) is coated with a micro-layer of a piezoelectric material, such as piezoelectric polydimethylsiloxane (PE-PDMS). In some embodiments, cantilevers can be precut into the base layer, thereby forming piezoelectric cantilever sensors. As the cardiomyocytes begin synchronous contraction (i.e. as the cantilevers move), the cellular mechanical energy is then converted to electrical energy via the PE-PDMS. By measuring the electrical output of the PE-PDMS, the cell-culture platform is able to quantify and output a numeric output to quantify the cells' contractile strength.

Simultaneously, the movement of the cantilevers can also be visualized to calculate the contractile force exerted by the cells. Two simultaneous measurements of contractile output can be obtained from a single platform, increasing robustness of data. The contractile forces of the cardiomyocytes are similar to that in vivo, thus making the cell-culture platform a useful tool in studying the effects of various drugs on the human heart. These chips can be manufactured and kept at room temperature for an indefinite amount of time before seeding cells and further use.

Currently, there are no other existing multi-chamber benchtop microphysiological platforms (i.e., organ-on-a-chip devices) that mimic the physiology and function of the heart muscle that also allow for organ function to be measured piezoelectrically. The data generated from the cell-culture platform described in the present disclosure provides a foundational understanding of the contractile strength differences between healthy and diseased cardiomyocytes.

Additionally, the cell-culture platform, configured as a heart-on-chip platform, can serve as a platform for the discovery of potential new therapeutics for cardiac disease, such as muscular dystrophy. The use of human cells to engineer the heart-on-chip platform also is an advantage over preclinical, animal-based approaches. This platform can be used to study physiological differences in how diseases present and how drugs affect different people groups. For example, male versus female responses to standard cardiovascular treatment drugs. Furthermore, various ethnic groups' cells can be seeded onto this platform to examine various disease contractility data as well as responses to standard drug treatment. Advantageously, the heart-on-chip platform can be adapted to custom-build patient-specific devices with various disease phenotypes for the purpose of drug safety and efficacy testing-personalized medicine.

In some embodiments, the organ-on-chip platform can also be adapted for other cell types to study other organs and diseases, such as, but not limited to diseases affecting skeletal and smooth muscle cells.

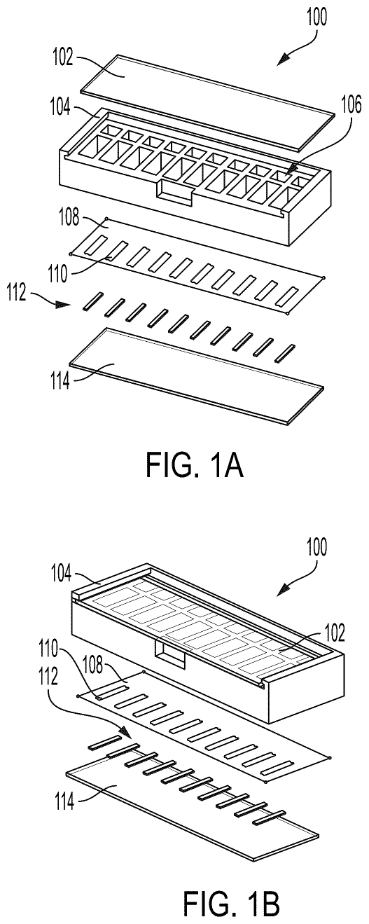

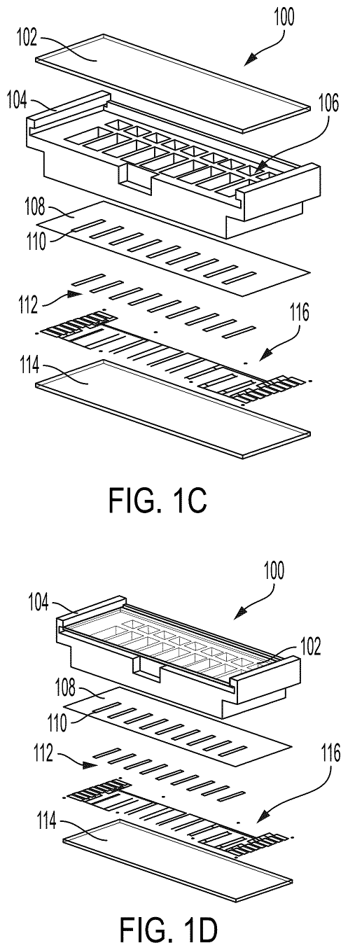

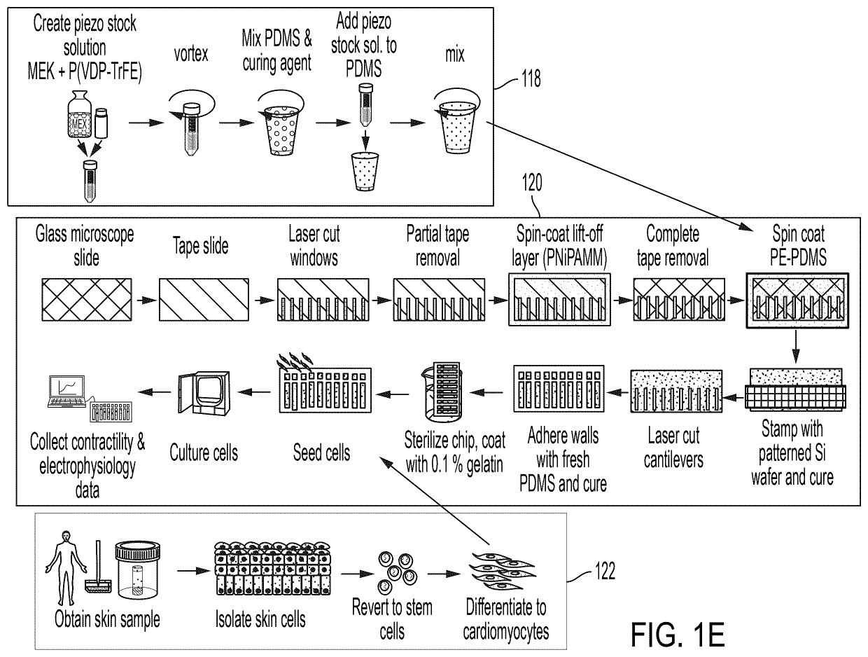

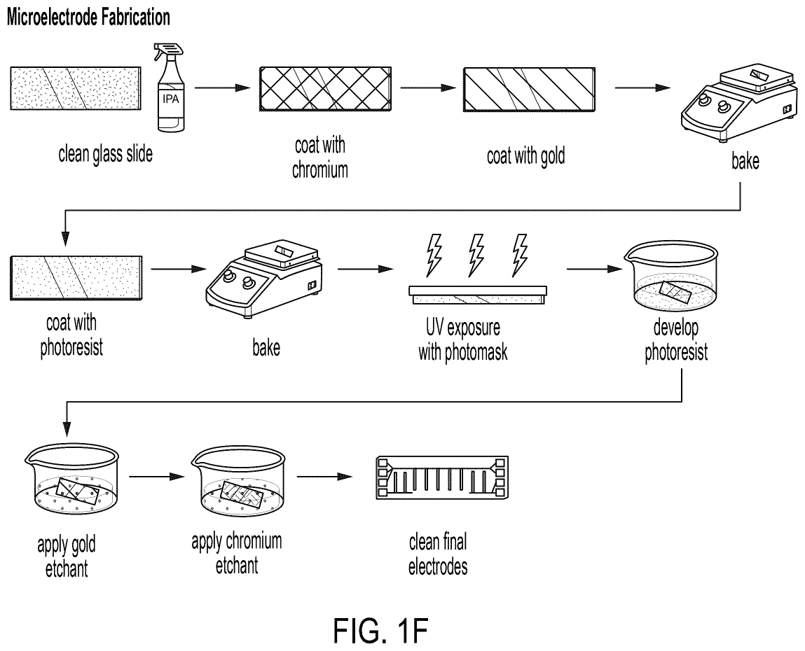

FIGS. 1A and 1B show one example according to the present disclosure. The cell-culture device 100 represents a heart-on-chip device that is a multi-chambered, cell co-culture platform including a plurality of distinct sections divided by walls 104. Each section of the cell-culture device 100 has a chamber for culturing cardiomyocytes or other muscle cells (e.g., skeletal muscle, smooth muscle) Each section may include one or more piezoelectric-polydimethylsiloxane (PE-PDMS) cantilevers. The sections may also be divided to include a coculture chamber that shares media with the cardiomyocyte (or other muscle cell) culture chamber.

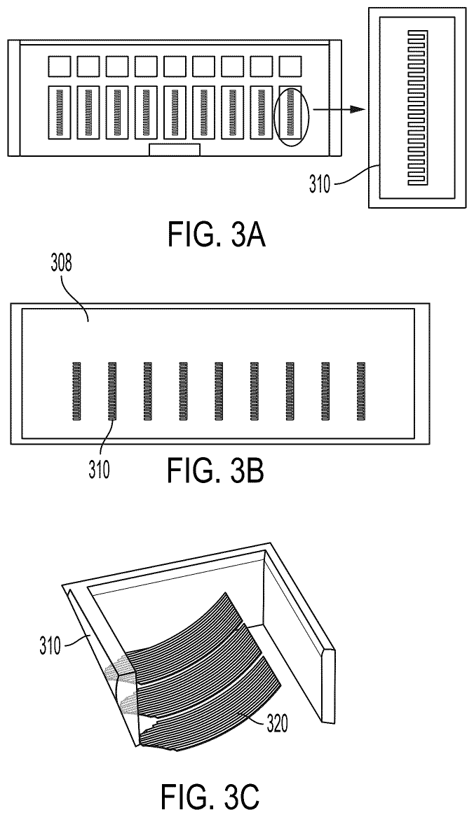

The heart-on-chip device 100 includes a cover 102, such as a glass slide, placed on top of walls 104 that define multiple chambers that each are divided into a pair of compartments 106 where the cells will be seeded. As an example, the walls may be composed of PDMS or other similar materials. The walls 104 sit on top of a PE-PDMS micro-layer 108 with sections 110 in one compartment 106 of each chamber. The sections 110 will be further described in FIGS. 3A and 3B. The PE-PDMS micro-layer 108 is coated, deposited, or otherwise coupled to a base layer 114. As one example, the base layer may be a glass slide. A poly(N-isopropylacrylamide) (PNiPAM) micro-layer 112 can be coated on the base layer 114 to prevent the piezo-PDMS micro-layer 108 at sections 110 from adhering to the base layer 114.

As will be described, each chamber or section 106 can include one or more piezoelectric sensors formed by the PE-PDMS micro-layer 108. As one example, each chamber or section 106 can include a single section 110, which may be formed as a single cantilever coated with or otherwise coupled to the PE-PDMS micro-layer 108. Alternatively, a chamber or section 106 may include more than one section 110, such as by forming a plurality of cantilevers that are coated or otherwise coupled to the PE-PDMS micro-layer 108.

FIGS. 1C-1D show the cell-culture device 100 including microelectrodes 116 embedded on the base layer 114 and positioned beneath the PE-PDMS micro-layer 108 and configured to detect deflections of cantilevers. The deflection signal is transmitted to the signal to processor. In a non-limiting example, the microelectrodes 116 may be gold microelectrodes.

The piezoelectric sensors (i.e., the PE-PDMS microlayer 108 and microelectrodes 116) are placed into electrical communication with a processor for measuring electrical data from the piezoelectric sensors. The processor may be local to, or remote from, the cell-culture device 100. For instance, the processor may be integrated with the cell-culture device 100. As an example, the cell-culture device 100 may include a printed circuit board (“PCB”) that may include a processor coupled thereto. The PCB may be configured such that it spans the base layer 114 of the cell-culture device 100, and such that it is aligned with the piezoelectric sensors (e.g., such that the PCB is aligned with individual cantilevers that may be formed).

The piezoelectric sensors may be connected to the processor via a wired connection, a wireless connection, or both. In those instances where multiple cantilevers are formed in a cell-culture device 100, each cantilever can be individually wired to the processor such that electrical data can be individually measured from each chamber as a summation of electrical data before reaching the processor.

FIG. 1E illustrates an example workflow for constructing a cell-culture chip according to some embodiments described in the present disclosure, and for seeding that cell-culture chip with cardiomyocytes. As illustrated, a PE-PDMS material is manufactured 118 by creating a piezo stock solution by combining MEK and P(VDP-TrFE). The resulting solution is vortexed. PDMS and a curing agent are mixed. The piezo stock solution is added to and mixed with the PDMS mixture, which is then used to coat the chip and/or sensor substrate.

A substrate is selected for the cell-culture chip 120, which as illustrated can include a glass microscope slide. The substrate is taped, and windows are laser cut into the substrate. The tape portions covering the windows are removed, and a lift-off layer (e.g., PNiPAM) is spin-coated onto the substrate. The remainder of the tape is removed, and the prepared PE-PDMS mixture is spin-coated onto the substrate. The PE-PDMS coated substrate is stamped with a patterned silicon wafer and cured in a 37° C. oven overnight. Alternatively, instead of applying a flat coating during the coating step, microcontact printing can be performed, whereby the coating is applied with a stamp containing small grooves to create ridges for cells to attach into and align properly. Cantilevers are then laser cut into the substrate. Walls are adhered to the substrate to form the cell culture sections. The walls can be adhered with fresh PDMS and cured in a 37° C. overnight.

The chip can then be sterilized and coated with 0.1% gelatin. Cells can be seeded in the sterilized chip. For example, as illustrated, cardiomyocytes can be seeded either alone, or with one or more co-cultured cell type(s) if desired. As a non-limiting example, cardiomyocytes can be obtained by collecting a skin sample from a subject 122, isolating skin cells, reverting the skin cells to stem cells, and differentiating the stem cells to cardiomyocytes. The cells can be cultured for eight or more days and then analyzed as described in the present disclosure, such as by collecting contractility and/or electrophysiology data from the cultured cells. In a non-limiting example, the cardiomyocytes can be cultured until contraction is detected, which may be as early as 8 days after seeding. Further, the cells may be cultured up to or beyond 28 days. In a non-limiting example, continuous contractility measurements can be taken during cell culture until the culture is ready to be terminated for endpoint assays.

FIG. 1F shows the microelectrode fabrication method of glass slides used as a substrate in the cell-culture chip of FIGS. 1C-1D. In a non-limiting example, the glass slide base is cleaned, such as with 70% isopropyl alcohol in a sonicator and RCA Organic Cleaning Kits. The glass slide is then fixed into a thermal evaporator and coated with a 5-10 nm layer of chromium using chromium-coated tungsten rods. Chromium acts as an adhesive layer since it easily attaches to glass. Next, the thermal evaporator is used to deposit a 50-100 nm layer of gold. The gold-deposited glass slide is then baked to dehydrate and remove moisture. Thereafter the coated glass slides are spin-coated with AZ-4330 photoresist at 500 rpm for 4 seconds. The photoresist coating is then soft baked at 110° C. for 80 seconds in direct contact with a hotplate. Thereafter, the coated glass slide is placed in an aligner where it is patterned using a custom-designed mylar film photomask. The glass slide is then exposed to UV light at a wavelength of 310-450 nm. The glass slide is then removed from the aligner and the photoresist is developed. The exposed gold layer is wet etched using a gold etchant and rinsed with acetone. The chromium is wet etched using a chromium etchant. The resulting glass slide is cleaned and sterilized thoroughly to be used as the substrate in the cell-culture chip described above.

In one example construction, a 24×76 mm glass microscope slide is used for the base layer 104 of the heart-on-chip device 100. Tape is placed over the slide and trimmed on the edges using a scalpel. Windows are laser cut into the tape, but not through the glass using the following settings: autofocus, vector mode, 5 speed, 5% power, and 2500 frequency. These windows are 11 mm×1.4 mm and are centered in each of the cardiomyocyte chambers. Once laser cut, the tape within these small windows is removed using high-precision tweezers. A 10% poly(Nisopropylacrylamide) (PNiPAM) solution is spin-coated into this removed-window region only. This solution prevents the PE-PDMS solution from adhering to the glass at sections 110. All the tape is then removed. A PE-PDMS solution, is then spin-coated over the entire chip. The chips are left to cure in a 37° C. oven for 16 hours.

At sections 110, 18 cantilevers, such as those depicted in FIG. 3, measuring 0.3 mm×1.2 mm, are laser-cut in each window section using the following settings: autofocus, vector mode, 3 speed, 3% power, and 2500 frequency. These base platforms can be stored at room temperature for an indefinite amount of time. When ready to use, the platforms are sonicated in 50% ethanol for 15 minutes and then sterilized using ethylene oxide.

As noted above, walls 104 divide the plurality of chambers (and thus the pairs of co-cultured compartments 106). These walls may be 18 mm tall and 2 mm thick and may be composed of PDMS or other suitable materials. Each section contains a main cardiomyocyte chamber (13 mm×5.4 mm) and a co-culture chamber for another or multiple cell types (5 mm×5.4 mm).

As an example method for manufacturing the walls 104 of the cell-culture device 100, walls may be 3D printed using acrylonitrile butadiene styrene (ABS).

To adhere the walls to the chip, the sterile chip and sterile walls are opened and dried in the cell culture hood. Fresh PDMS is carefully placed only on one side of the walls. The walls are aligned by eye to the base slide. The walls are pressed on with a thin layer of 10:1 PDMS and curing agent and kept in a sterile box in the 37° C. oven for 16 hours.

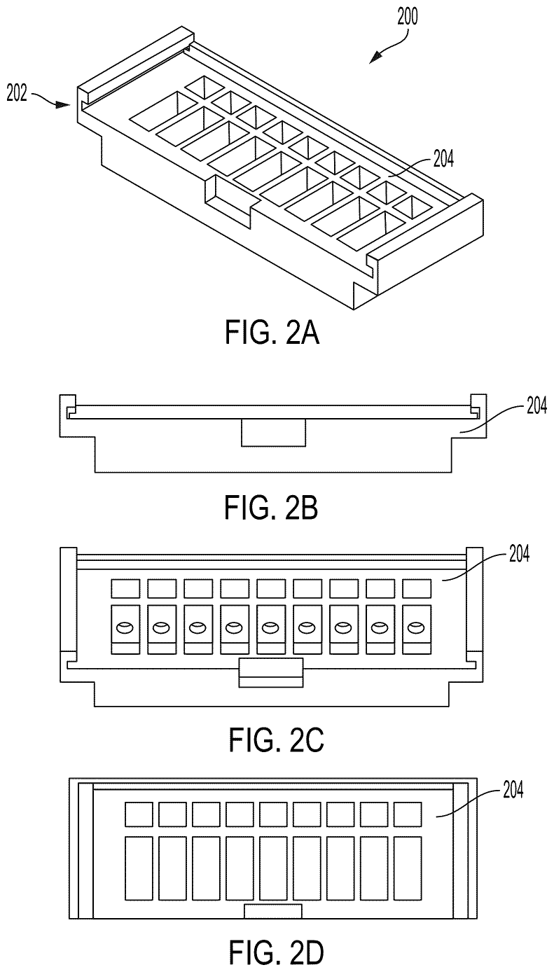

In another non-limiting example, a wall design is shown in FIG. 2 using 3D printed walls instead of PDMS molded walls. This alternative design can maintain the inner chamber dimensions described above. In some embodiments, the walls 204 created using a 3D printing, or other additive manufacturing technique, can be made thicker than when using the molding technique described above. For instance, the outer walls can be created to be 4 mm instead of the original 2 mm. Additionally or alternatively, the walls 204 can be made shorter than when using molding techniques. For instance, the wall design can be 10 mm tall instead of 18 mm.

The embodiment of the cell-culture device 200 illustrated in FIG. 2 includes lips 202 formed on the top end of one or more of the walls 204 (e.g., two of the walls 204 in the illustrated embodiment), allowing for the top glass slide to be received and held into place by the lips 202, rather than being adhered to the top surface of the cell-culture device 200. The media can be introduced to the cell culture chambers by removing the top glass slide to provide access to the cell culture chambers. In building the lips 202 for the glass slide to be removed by pulling across each section rather than over all of the sections, the risk of cross contamination between sections is reduced. Having a top to the cell-culture device 200 reduces risk of contamination, while the ability to remove this top slide enhances imaging capabilities. Media changes and cell seeding may be more accessible with this design.

According to aspects of the present disclosure, the piezoelectric material used for forming the piezoelectric micro-layer 108 may be composed of piezoelectric-polydimethylsiloxane (PE-PDMS). The PE-PDMS material can be created by blending a piezoelectric substance with a solvent and a substrate material. For example, the substrate material may be a silicone elastomer base mixed with a curing agent.

As one example, the piezoelectric substance is polyvinylidene fluoride-trifluoroethylene (P(VDF-TrFE)). An example solvent that can be used is methyl-ethylketone (MEK). In a non-limiting example, the concentration of PVDF to TrFE may be 70/30. An alternative concentration may be 50/50 which results in a high-piezo and high-sensitivity mixture. Alternatively, poly(vinylidene fluoride-co-hexafluoropropylene) (P(VDF-HFP)) may be employed as a piezoelectric material. P(VDF-HFP) may be dissolved in n-methyl-2-pyrrolidine (NMP), dimethylformamide (DMF), or dimethylacetamide (DMAc).

By blending the piezoelectric substance in different percentages of the volume of PE-PDMS material (70/30 P(VDF-TrFE) mixture) or xpz-PDMS material (50/50 P(VDF-TrFE) mixture), the sensitivity of the piezoelectric material can be adjusted. As a non-limiting example, the percentage of piezoelectric substance in the total volume of PE-PDMS may be selected from the range of 1-10%, or subranges therewithin (e.g., between 1 and 7%). Higher or lower percentages may also be employed. As noted above, different percentage blends will result in piezoelectric materials with different sensitivities. Sensitivity can be tailored to a desired cell type. For example, cardiac muscle cells, skeletal muscle cells, and smooth muscle cells have high, moderate, and low contractile strength, respectively. Since cardiac muscle cells have higher contractile strength, a less sensitive PE-PDMS can be used as compared to the PE-PDMS used for those muscle cell types with lower contractile strength. As a non-limiting example, a 1% PE-PDMS can be used with cardiac muscle cells, whereas a higher sensitivity PE-PDMS formulation, such as 10% PE-PDMS, may be needed for cells such as blood vessel cells and smooth muscle cells, which have a lower contractile strength than cardiac muscle cells. Higher sensitivity PE-PDMS can also be used. Unlike other piezoelectric materials that use P(VDF-TrFE), the PE-PDMS material described in the present disclosure is formed by blending the P(VDF-TrFE) with a substrate material, rather than forming scaffolds of P(VDF-TrFE) or depositing a film of P(VDF-TrFE) on a substrate.

By combining the P(VDF-TrFE) in a powder form with the solvent (e.g., MEK), the powder can be dissolved, thereby creating a gel-like solution. A 10:1 ratio of PDMS and curing agent can be added to this gel-like structure and combined using a planetary mixer and degasser.

Surface modifications to the PE-PDMS may be performed. The material can be submitted to chemical annealing via an MEK vapor bath for varying amounts of time before or after curing. For instance, 30 to 90 minutes or longer in a vapor bath can decrease the size of P(VDF-TrFE) particles lending a more homogenous, smooth surface. During the curing process, patterning of the material can be implemented through micro-contact molding. Creating patterns on the surface of the material, such as micro-ridges, can enhance cell adhesion.

Referring now to FIGS. 3A-3C, the PE-PDMS microlayer 308 at sections 310 may be laser cut into a plurality of cantilevers 320 that move freely in response to cellular forces.

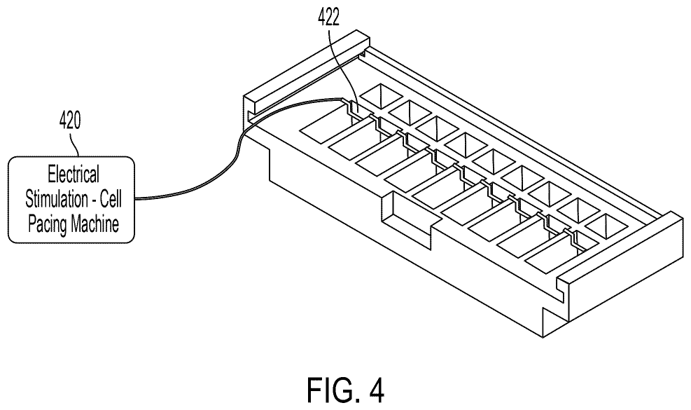

FIG. 4 illustrates an example cell pacing machine setup, including an electrical stimulation source 420 and electrodes 422 for providing electrical stimulation to the seeded cells. To encourage synchronous contractions in the cardiomyocytes before analysis, a custom-shaped electrode 402 can be placed into each well containing cardiomyocytes. This electrode may be configured to avoid contacting the PE-PDMS base of the chip, by only having a section of the electrode submerged in each well's media. The cells can be stimulated by releasing a small cyclical voltage across each of the cardiomyocyte wells for only a short period of time. Once the cells begin beating in sync, the stimulation may be halted and the contractility measurement system may be used to gather the unheeded organic cell-PE-PDMS interaction voltage output.

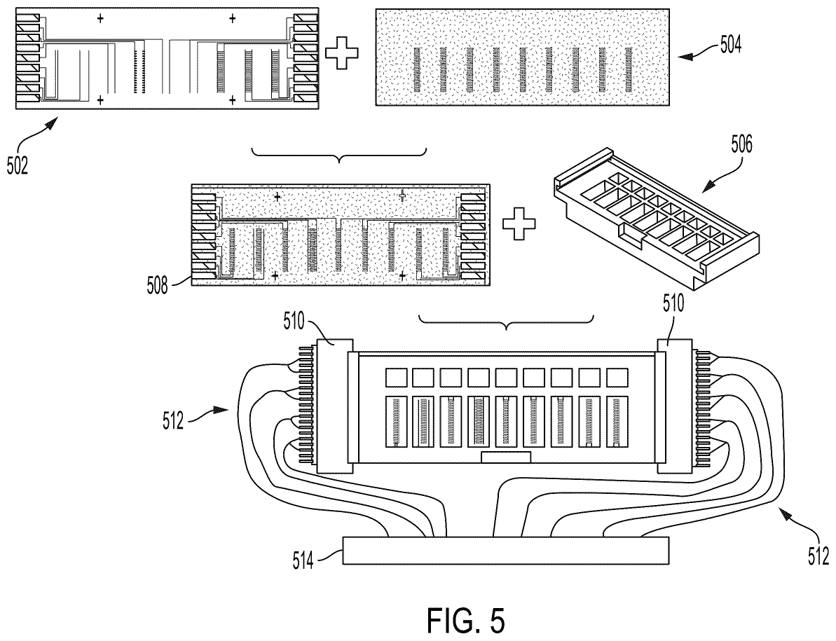

The contractile force measurement system is shown in FIG. 5. The glass slide with the microelectrode array 502 is spin-coated with PE-PDMS and left at room temperature or in a 37° C. oven to cure overnight. Cantilevers are laser cut into the PE-PDMS 504. The 3D printed walls 506 are adhered, leaving exposed the gold electrode pads 508. Edge connectors 510 are clipped onto each side of the base glass slide allowing the electrical output from the gold electrode pads to be transmitted via connected wires 512 to the processor 514. The voltage and time are collected and stored. As in the cell pacing example, the cardiomyocytes beat in sync, whereupon the mechanical energy is converted to electrical energy via the PE-PDMS substrate. This electrical signal may be output as a single voltage by using a positive and negative electrode pair within each well. Alternatively, each set of electrode pair may be connected to an Engineering Laboratory Virtual Instrumentation Suite (ELVIS) board, which will feed the data, in real-time, into a computer 524 to be processed.

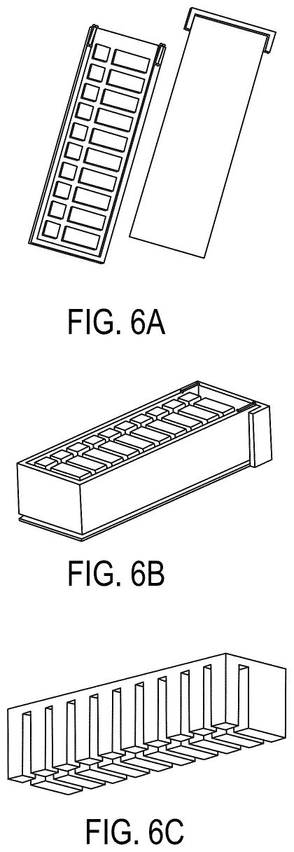

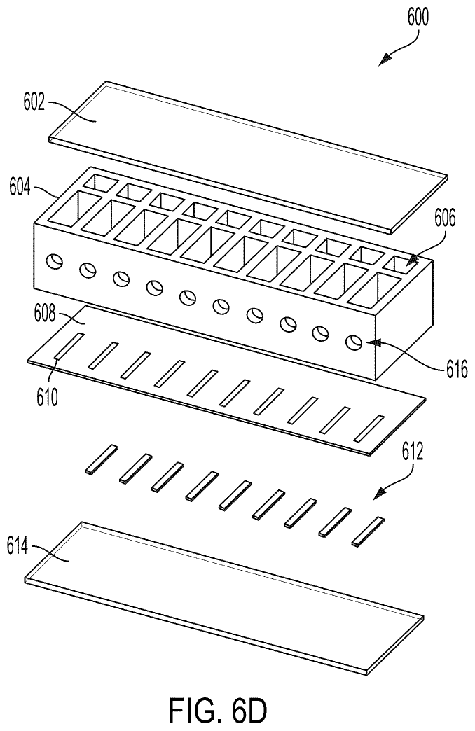

Referring to FIG. 6, the two-part wall mold depicted in FIGS. 6A-6B is joined and sealed using this PDMS. The two pieces are placed into a 70° C. oven for 20 minutes. Canola oil is sprayed into the mold generously. This is kept in a 60 psi vacuum for 20 minutes. The molds are then flipped upside-down, with one end propped up for an additional 10 minutes in the vacuum to drain excess oil. Fresh PDMS is poured into the mold. The filled mold is placed in a bell-vacuum for five minutes to ensure there are no air pockets in the mold. The vacuum is shut off for two minutes to allow for surface bubbles to pop. This bell vacuum cycle is repeated three times. The filled mold is then moved to the 60 psi vacuum for 45 minutes. Finally, the mold is placed in a 37° C. oven for 16 hours. The walls are carefully demolded by using a scalpel to cut along the outside edges of the mold. The end piece is carefully removed. A long tweezer is then used to carefully lift the walls from the mold.

FIG. 6D illustrates an alternate chip design used molded walls, wherein holes 616 are drilled through each chamber of the cell-culture device 600. As an example, the holes 616 may have a diameter on the order of 1 mm. To drill the holes 616, a 3D printed drill support can be inserted into the chambers. A non-limiting example of the 3D printed drill support is shown in FIG. 6C. A 1 mm drill-bit is used to drill through all three walls of each section. One-eighth inch fittings are then inserted into each of the outside 20 holes for easy media changes. The hole in the middle wall is utilized to share media between the two chambers for each co-culture section. The walls are sterilized by placing them in 50% ethanol in the sonicator for 15 minutes. Once air-dried, the walls are autoclaved or treated with ethylene oxide for sterilization.

Because PDMS is hydrophobic, which could cause an issue when testing hydrophobic drugs, in some embodiments the cell-culture device can be composed of materials other than PDMS. For example, the cull-culture device can be composed of a resin that is manufactured using 3D printing or other additive manufacturing techniques, which can be advantageous when a non-hydrophobic material design is preferred. The base of the chip where cells will be seeded can also be coated with PDMS alternatives, such as 10% gelatin, combined with P(VDF-TrFE).

Additional treatments may be designed to be applied to the chip. For example, a micro-cardiac resynchronization therapy system may be designed and applied to the base of the chip throughout or immediately following cell culture. This would allow for the analysis of how different individuals or people groups respond to therapeutic treatments alone or in conjunction with various drugs.

As an example of using the cell-culture devices described in the present disclosure, after full-chip sterilization, human fibronectin is stamped into each cardiomyocyte chamber using standard micro-contact printing methods. In some embodiments, the fibronectin may be applied uniformly across the surface of the chambers (flat or micro-molded surface) or micro-contact printed to enhance cell alignment. Alternative coatings for cell adhesion, such as gelatin and Corning® Cell Tak™, may also be utilized. Cardiomyocytes are seeded into the main chamber of each section while fibroblasts are seeded onto the small chamber of each section. After at least eight days of continuous contractility measurements during culture, the cells are terminated for endpoint assays.

A live-dead assay may be performed to determine cell viability. A stain, such as the fluorescent stain DAPI, is used to stain cell nuclei. Alpha-actinin and phalloidin are used to identify cardiomyocyte maturity. In a non-limiting example other stains such as GATA4, Wheat Germ Agglutinin, cardiac troponin-T, and connexin-43 can be used to visualize morphology and give insights to protein expression of the cells.

In a non-limiting example, previously constructed micro-electrodes lie below the micro-layer PE-PDMS of each section. Edge connectors on each side of the chip allow a connection from the chip's electrodes to the micro-controller. Here, the data is captured and stored in an integrated memory chip. After the culture is terminated, the stored data can be downloaded onto a computer for processing. The electrical data are used to determine contractile force measurements and show differences between healthy and diseased myocytes' contractile strength. Other assays may be performed to examine additional characteristics of interest.

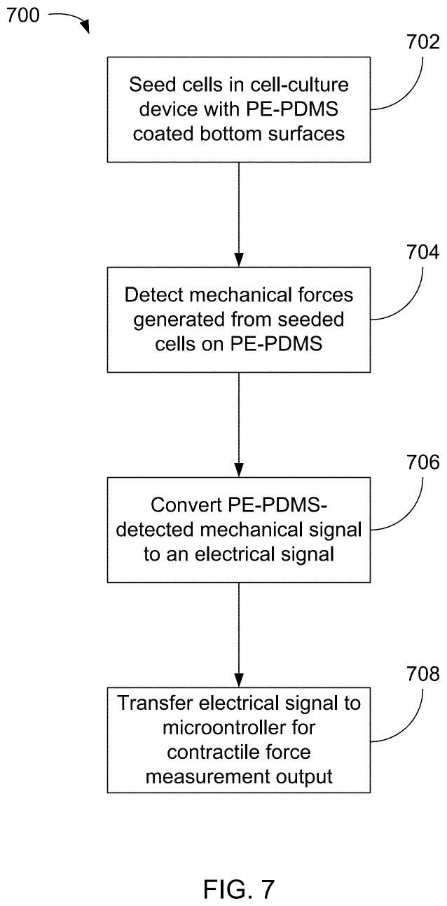

A non-limiting example of a method for measuring forces exerted by cells 700 is illustrated in FIG. 7. At step 702, cells are seeded in the chambers of the chip as described previously. At step 704, the mechanical forces generated by the cells act on the PE-PEDMS, whereupon at step 706, the mechanical forces are converted to an electrical signal. At step 708, the electrical signal is transferred to a microcontroller and processed for outputting a contractile force measurement.

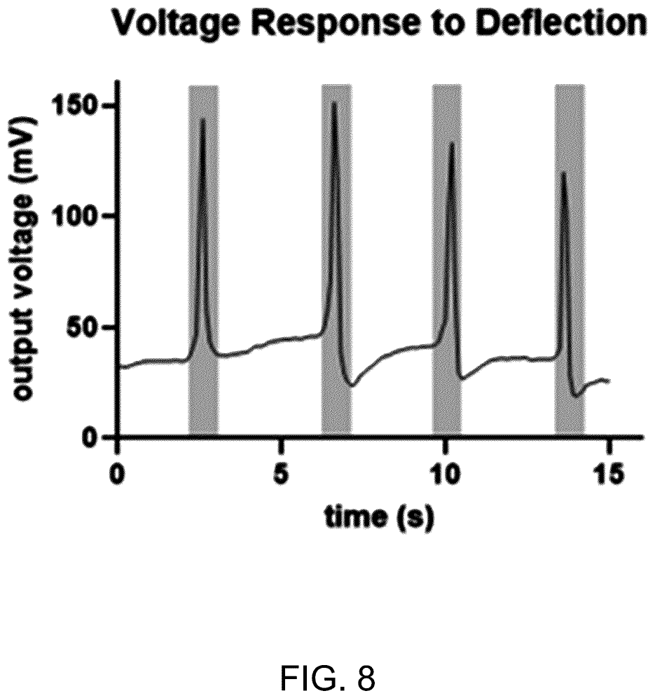

FIG. 8 illustrates example data of the voltage response of PE-PDMS material to manual substrate displacement. When displaced, a consistent electrical response can be recorded as a single voltage output. In a non-limiting example, voltage output of 6.5% P(VDF-TrFE) in PDMS over time is shown (the deflection period is shown in the shaded region).

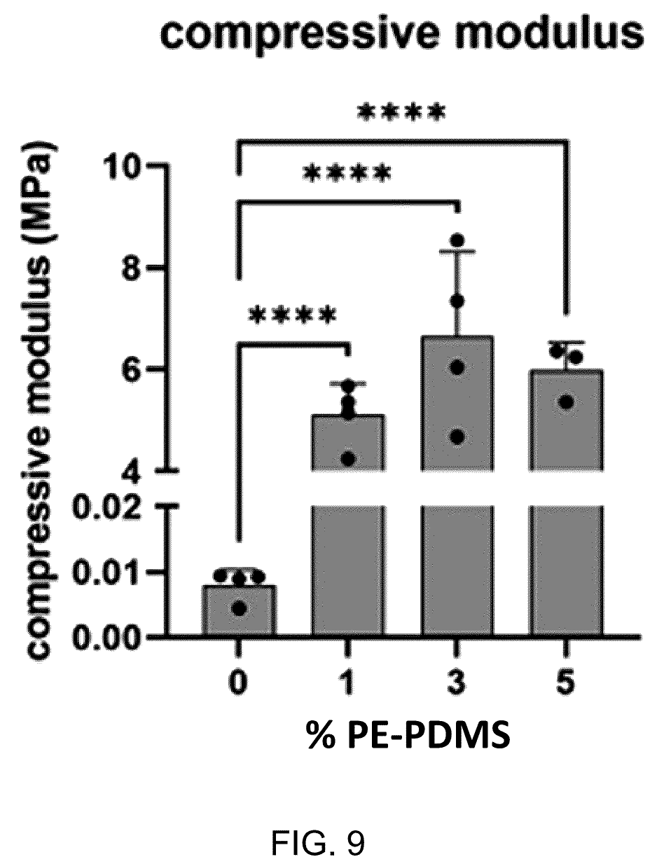

Referring to FIG. 9, example data of the stiffness of varying percentages of PE-PDMS material is shown. In a non-limiting example, the compressive modulus, or stiffness, is found using an Instron machine for uniaxial, unconfined compression testing. A higher compressive modulus correlates with a stiffer material. The example data in FIG. 9 suggests that the PE-PDMS substrate becomes significantly stiffer than the pure PDMS when the piezo substance is introduced.

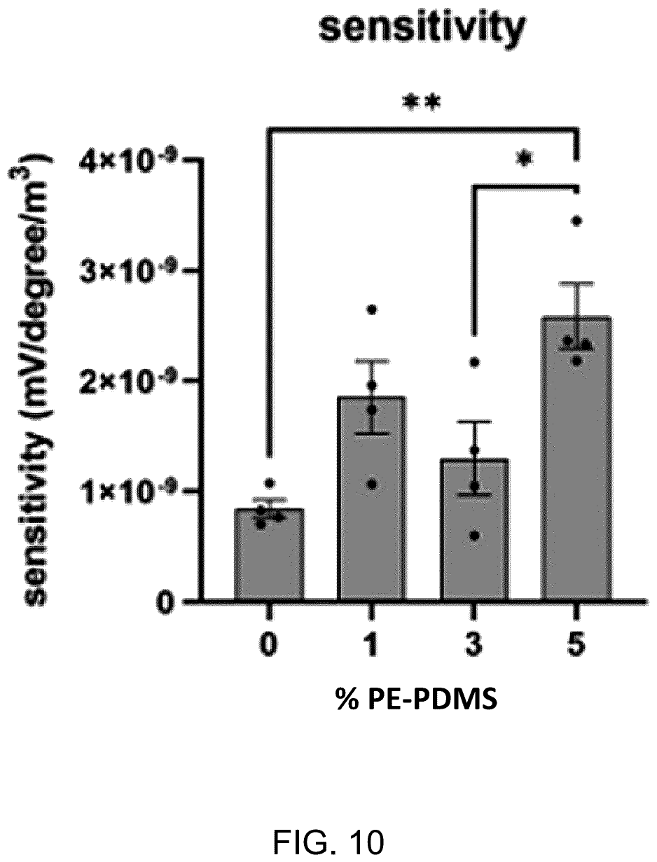

FIG. 10 shows example data of the sensitivity of varying percentages of PE-PDMS material. In a non-limiting example, PE-PDMS sensitivity is calculated as a representation of the contraction force-to-voltage ratio of varying percent PE-PDMS substrates. This sensitivity measurement is calculated as the voltage output in millivolts, per degree displacement, per volume PE-PDMS in cubic meters. A higher sensitivity coefficient correlates with a higher force-to-voltage ratio and a more sensitive material. The example data shown in FIG. 10 suggests that the PE-PDMS substrate becomes more sensitive than pure PDMS when the piezo substance is introduced, and that sensitivity increases nonlinearly as the percent P(VDF-TrFE) in PDMS increases.

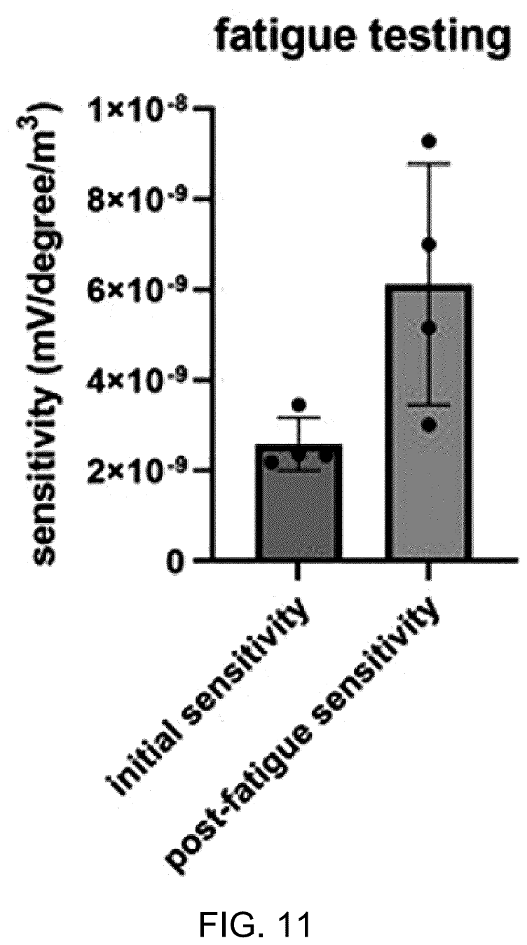

FIG. 11 shows initial sensitivity testing of 5% PE-PDMS. Samples were subjected to fatigue testing consisting of 37 displacements per minute, nonstop for seven days. This displacement rate was chosen because that is what has been shown with cardiomyocytes in in vitro cultures. After exactly seven days, samples were removed and tested for sensitivity again. There was no significant difference between sensitivity shown before and after fatigue testing, suggesting that the material would not lose sensitivity over time while in culture. This suggests that measurements collected throughout culture would be accurate, rather than a decreasing sensitivity of the cell culture substrate (i.e. PE-PDMS) that would otherwise require normalization, thus resulting in one fewer variable in the force measurement.

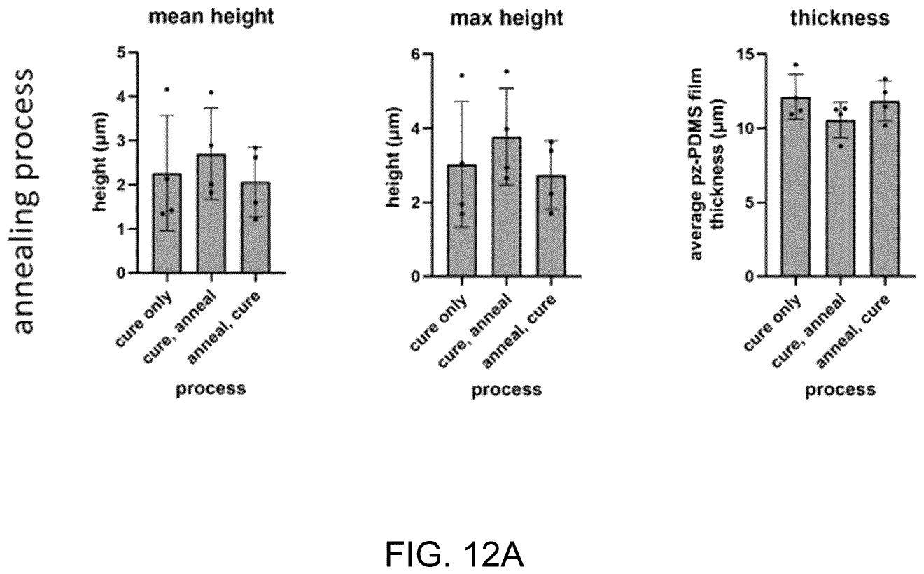

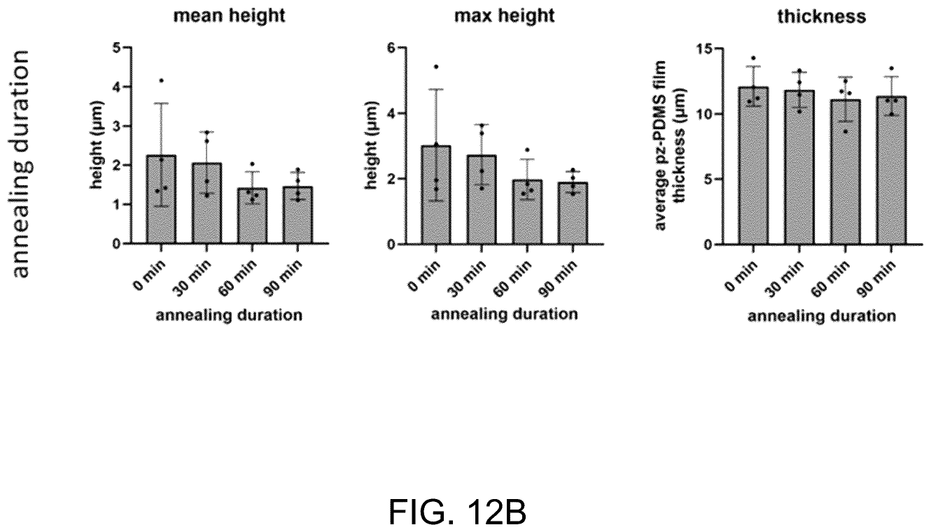

FIGS. 12A-12B show the resulting mean height, max height, and thickness of 5% PE-PDMS films spin-coated onto 15 mm glass coverslips. During chemical annealing, glass coverslips are placed in a MEK vapor bath. In FIG. 12A, the 5% PE-PDMS-coated glass coverslips were either not annealed, annealed for 30 minutes before curing, or annealed for 30 minutes after curing. In FIG. 12B, all 5% PE-PDMS coated glass coverslips were annealed after curing for 0 min, 30 min, 60 min, or 90 min. The results indicate that chemical annealing does not affect film thickness. Furthermore, chemical annealing after curing and for longer durations results in smaller piezo particles.

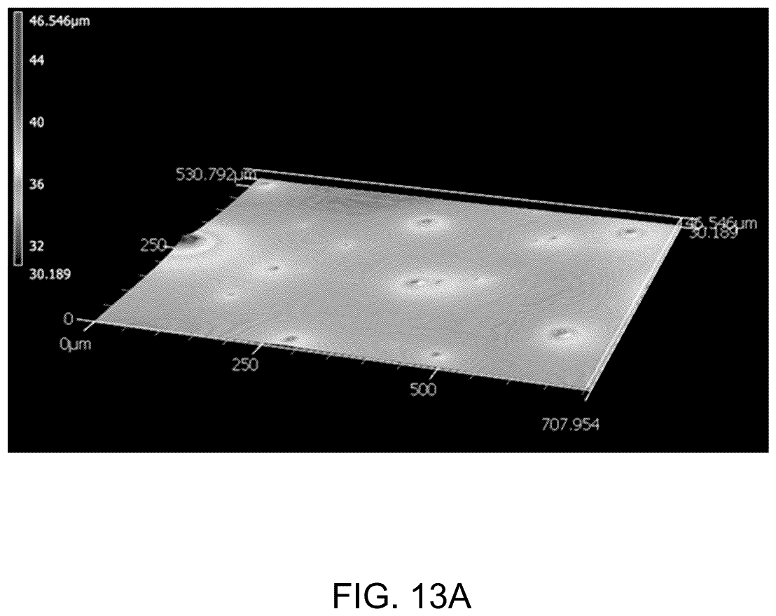

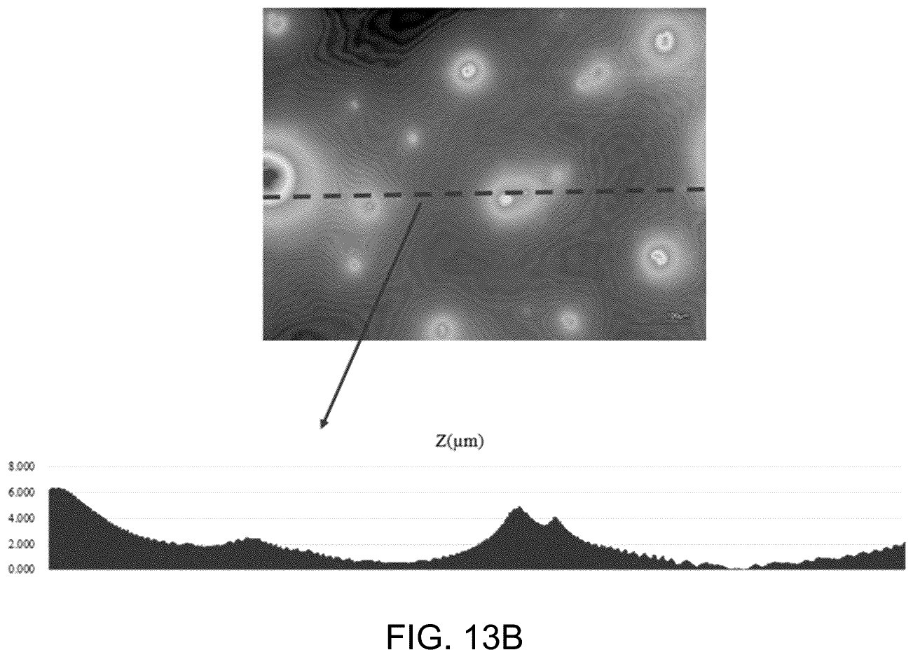

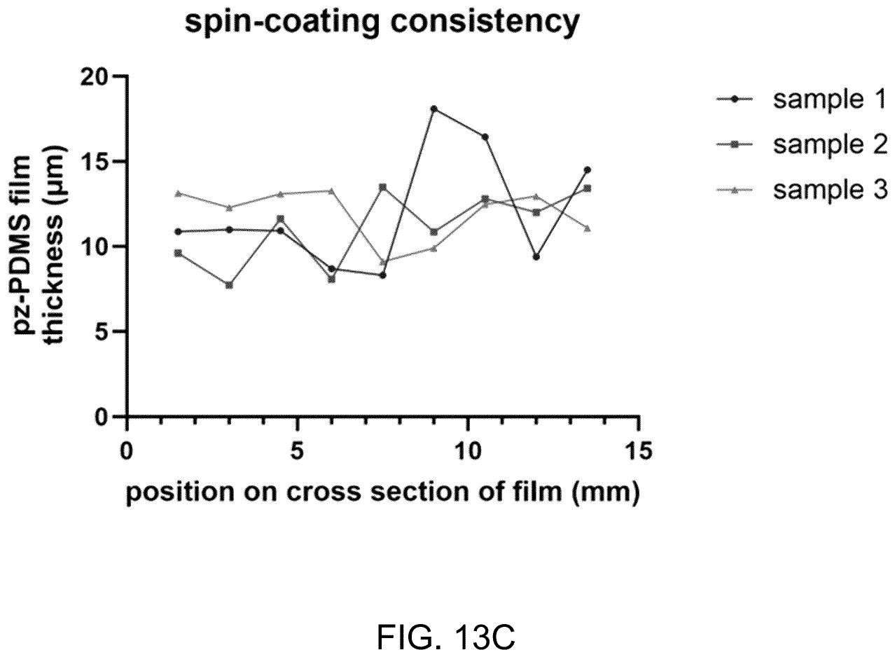

FIGS. 13A-13C show cross-sectional images and plot of 5% PE-PDMS films spin-coated onto 15 mm glass coverslips. A cross-section of the film is used to measure thickness of the film. The thicknesses of three separate spin-coated PE-PDMS samples on glass coverslips are shown in FIG. 13C, where nine measurements were taken across the middle of each coverslip and graphed. These results show that the spin-coating process with PE-PDMS results in a uniform, consistent microfilm.

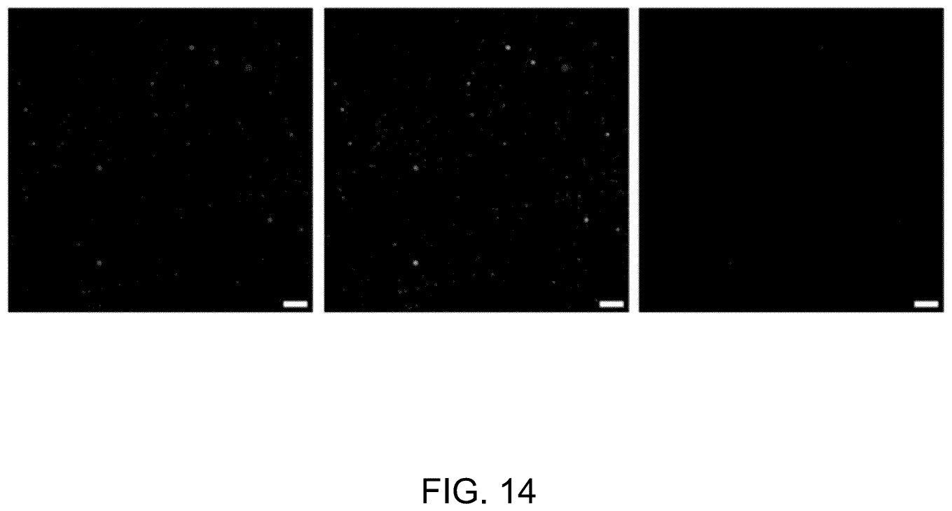

FIG. 14 shows the autofluorescence of PE-PDMS under fluorescence microscope, red channel at wavelength=594 nm (left), green channel at wavelength=488 nm (middle), and blue channel at wavelength=355 nm (right). No cells or other markers are included with the sample, which only contains fully-cured 5% PE-PDMS on a glass coverslip (spin-coated thickness 5-15 μm).

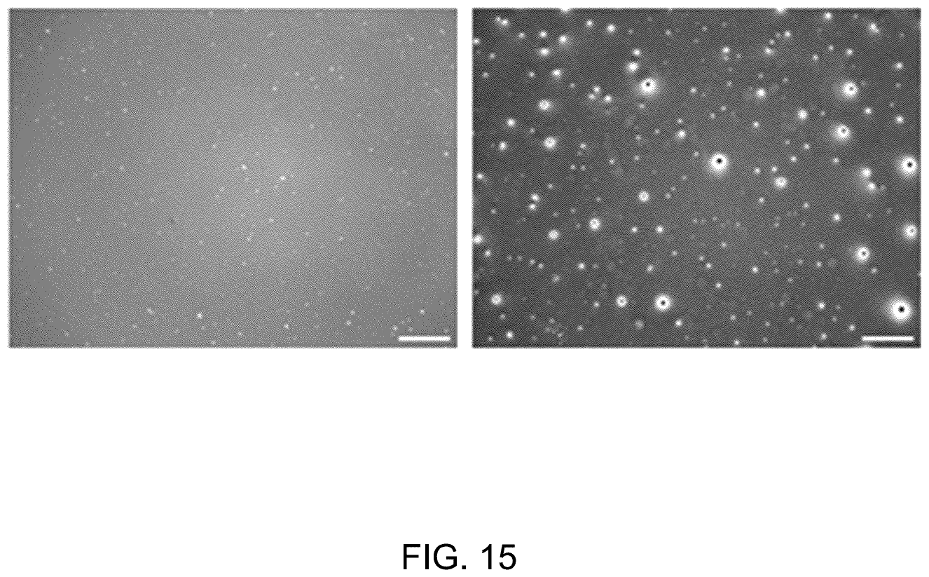

FIG. 15 shows phase/contrast images of pure PDMS (left) and PE-PDMS (right). The piezoelectric particles can be seen as bead-like structures throughout the surface of the PE-PDMS.

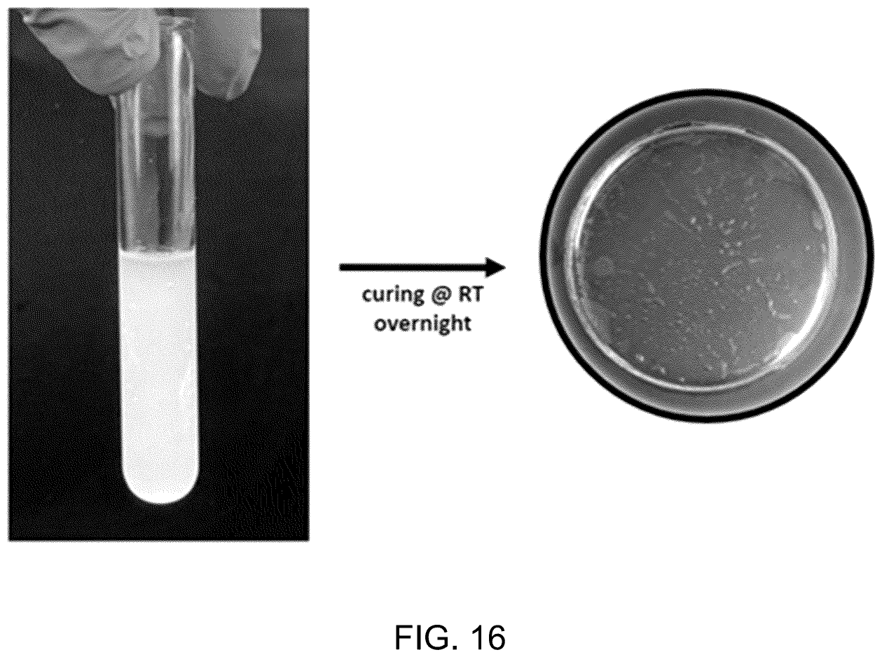

FIG. 16 depicts freshly mixed 3% PE-PDMS as a viscous liquid. After overnight curing at room temperature, the PE-PDMS is solid and clear.

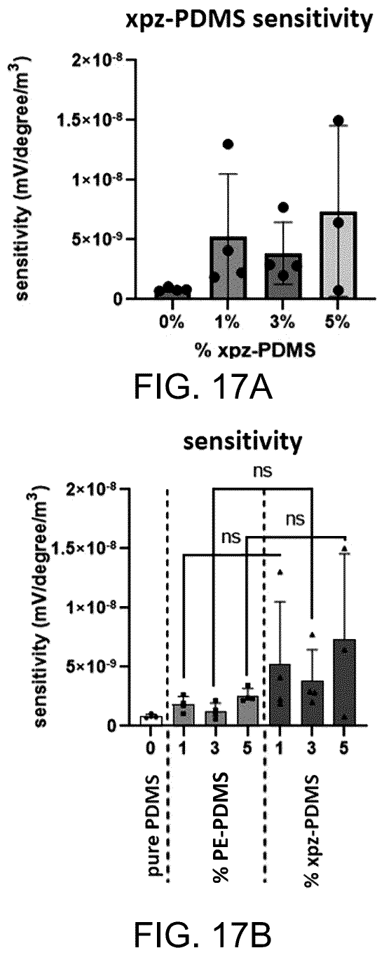

FIG. 17A illustrates the sensitivity results of xpz-PDMS (50/50 ratio of PVDF to TrFE) at 0%, 1%, 3%, and 5% xpz-PDMS concentrations. FIG. 17B shows that xpz-PDMS is more sensitive than PE-PDMS with 70/30 ratio of PVDF to TrFE.

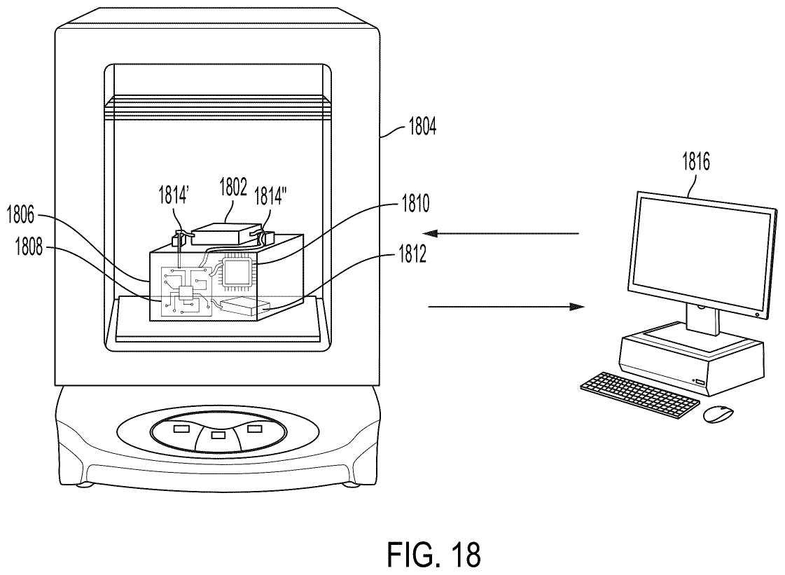

Referring now to FIG. 18, a mount 1802 is presented for the cell-culture chip to sit in within an incubator 1804. The mount 1802 includes a sterile tech box 1806 underneath that houses a microcontroller 1808 with an integrated memory chip 1810 and battery 1812. The edge connector will clip onto the chip, allowing for wires 1814′ and 1814″ to easily connect the cell-culture chip to the contained microcontroller 1808. This microcontroller 1808 will receive the electrical output data and automatically store this information, including time stamps, in the memory chip 1810. The memory chip 1810 can transmit the information to a nearby computer 1816 where the data can be visualized and processed or can be downloaded after the cell culture is terminated. The information may be transmitted wirelessly, such as via Bluetooth. Alternatively, the computer 1816 may be connected to the tech box 1806 via wired connection.

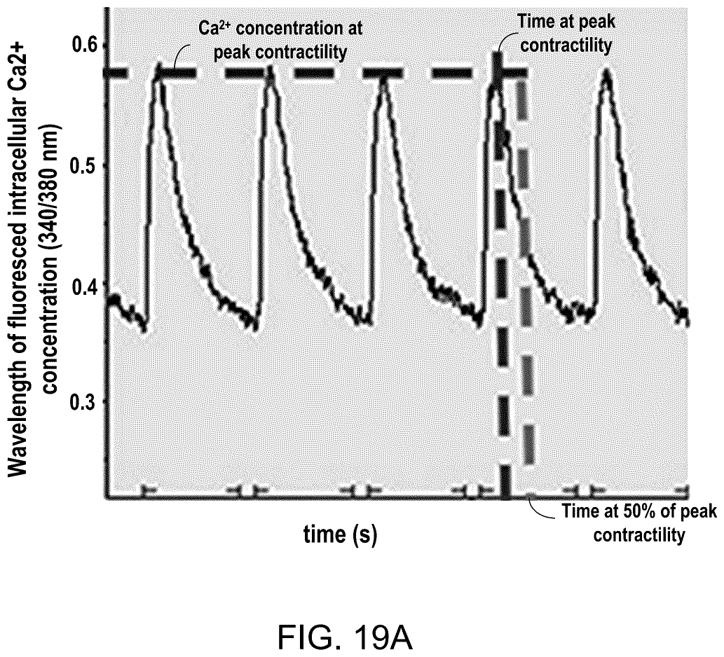

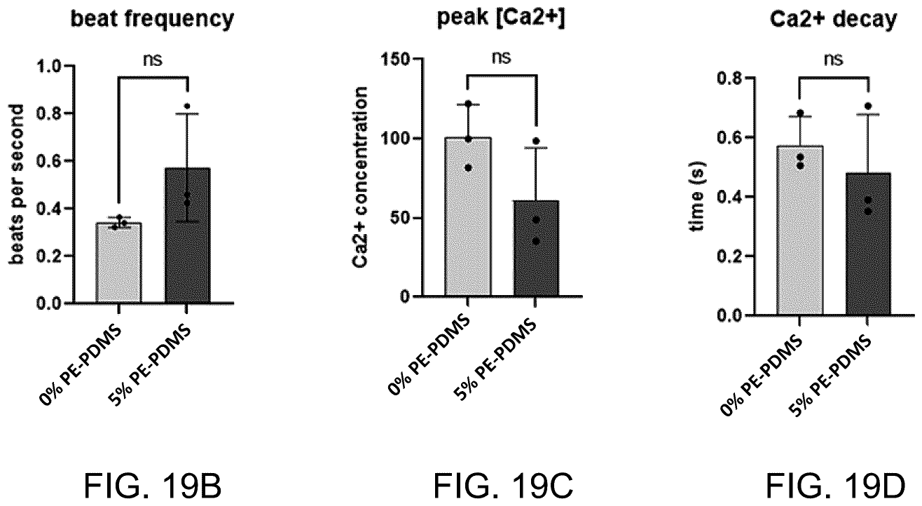

FIGS. 19A-19D provide results showing that cardiomyocytes seeded on pure PDMS and 5% PE-PDMS do not show significant differences in calcium cycling or beat frequency. FIG. 19B shows the results of the average beat frequency of cardiomyocytes. FIG. 19C shows the average peak Ca²⁺ concentration (single peak concentration marked on an example trace line in FIG. 19A). FIG. 19D shows the average time taken for the Ca²⁺ concentration to decay from its peak to 50% of the peak, calculated as the difference from time of peak concentration to the time 50% of the peak Ca²⁺ concentration was reached, as shown in FIG. 19A.

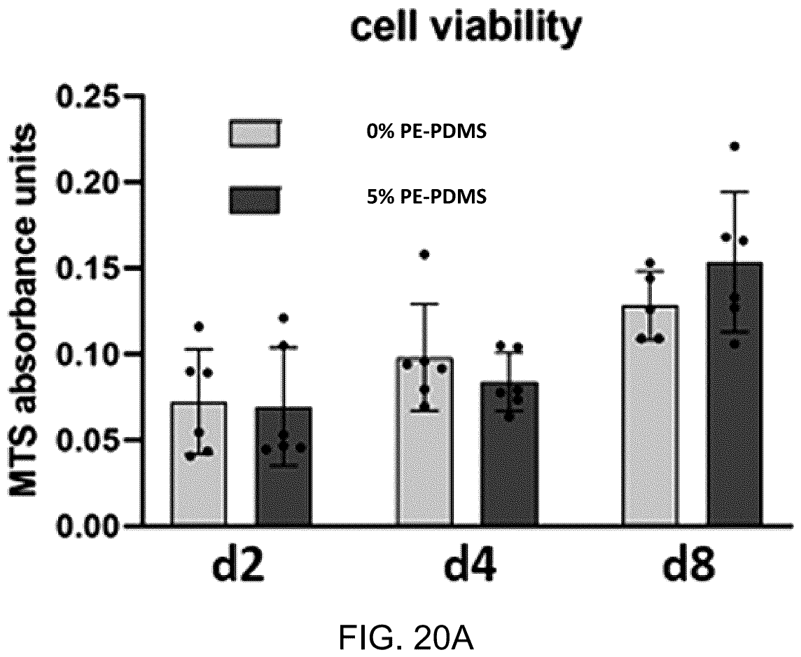

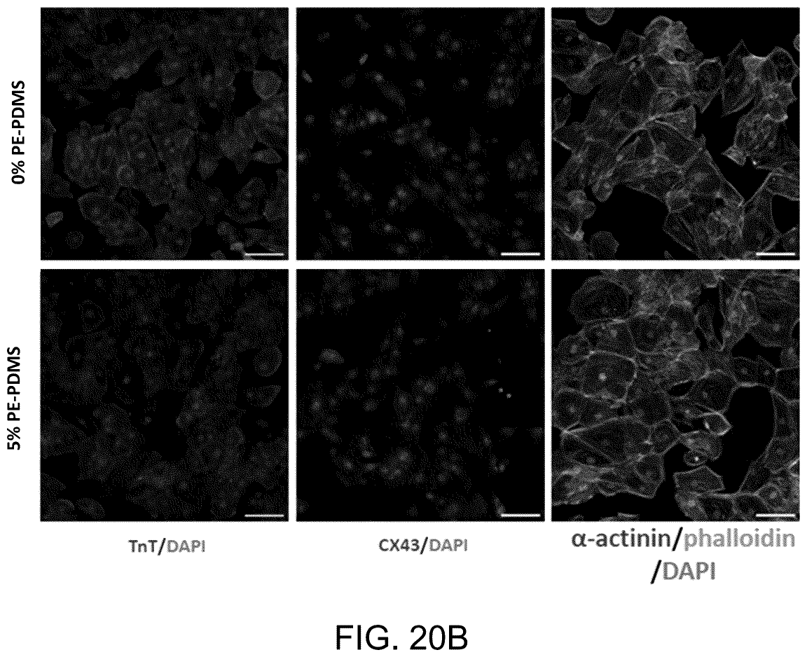

FIGS. 20A-20B show the results of the short-term biocompatibility shown by MTS assays and immunocytochemistry staining after 8 days of culture. In FIG. 20A, no significant difference in cell viability was observed for cardiomyocytes cultured on pure PDMS (0%) and PE-PDMS (5%). Furthermore, the immunocytochemistry staining after 8 days of culture shown in FIG. 20B shows mature, viable cardiomyocytes on 0% and 5% PE-PDMS substrates via the troponin T, alpha-actinin, connexin-43, phalloidin, and DAPI staining.

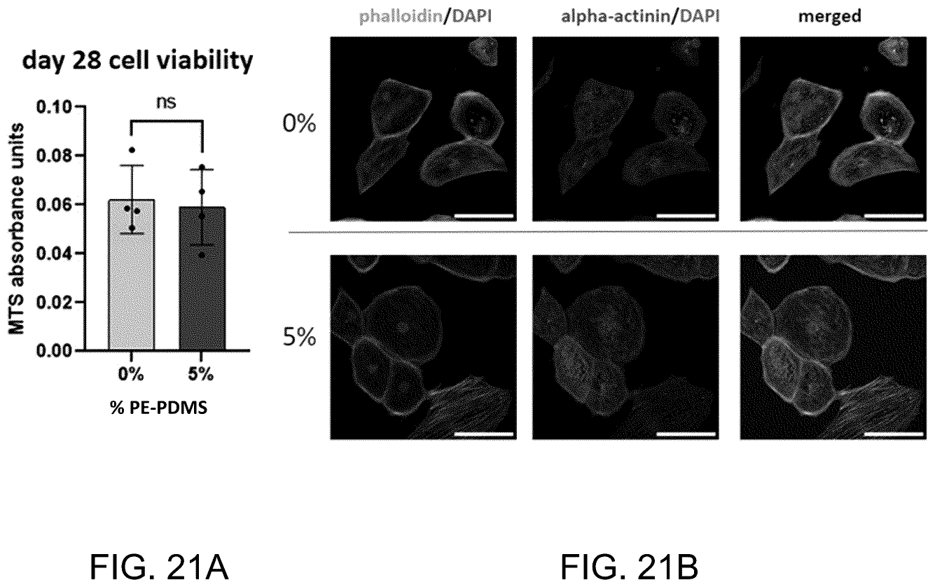

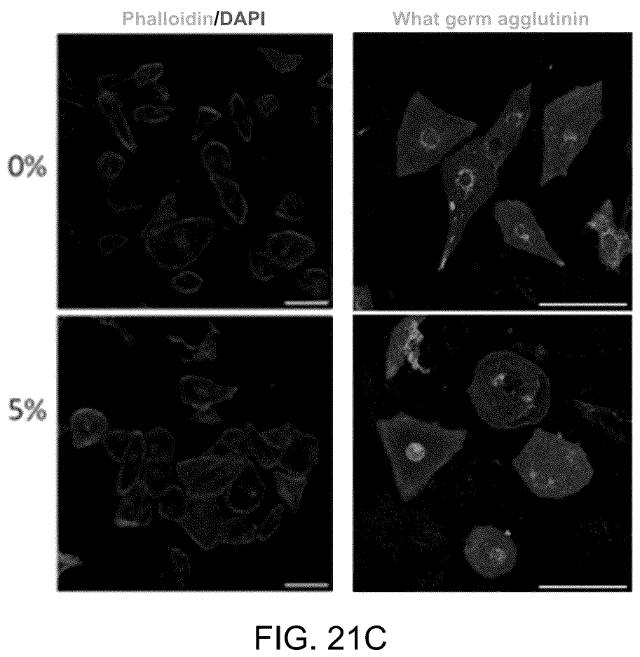

FIGS. 21A-21C indicate the long-term biocompatibility of cardiomyocyte cell culture on 0% and 5% PE-PDMS substrates shown by MTS assay and immunocytochemistry staining after 28 days of culture. In FIG. 21A, no significant difference in cell viability was observed for cardiomyocytes cultured on pure PDMS (0%) and PE-PDMS (5%). Furthermore, the immunocytochemistry staining after 28 days of culture shown in FIGS. 21B-21C show mature, viable cardiomyocytes on 0% and 5% PE-PDMS substrates via the alpha-actinin and phalloidin staining. The wheat germ agglutinin staining in FIG. 21C indicates that the cell morphologies are similar between cardiomyocytes cultured on pure PDMS and PE-PDMS (5%).

1. A cell-culture device for measuring forces exerted by cells, comprising:
a housing with one or more cell culture sections, wherein
each cell culture section includes one or more compartments for seeding one or more cell types, and
each compartment has a bottom surface that is coated with a layer of piezoelectric polydimethylsiloxane (PE-PDMS).

2. The cell-culture device of claim 1, further comprising a processor in electrical communication with each layer of PE-PDMS, wherein the processor is configured to measure a force detected on the PE-PDMS layer on the bottom surface of each compartment.

3. The cell-culture device of claim 1, wherein the PE-PDMS layer is cut into a plurality of cantilevers.

4. The cell-culture device of claim 3, wherein the plurality of cantilevers in each compartment have separate wirings to provide individual electrical signal outputs indicative of a contractile force applied to the layer of PE-PDMS.

5. The cell-culture device of claim 3, wherein each of the plurality of cantilevers is wire-connected to a printed circuit board (PCB).

6. The cell-culture device of claim 5, wherein the PCB detects an electrical signal from the plurality of cantilevers.

7. The cell-culture device of claim 6, further comprising a processor in electrical communication with the PCB, wherein the processor receives the electrical signal from the PCB and converts the electrical signal to a contractile force measurement.

8. The cell-culture device of claim 3, further comprising one or more microelectrodes embedded on a base layer and positioned beneath the PE-PDMS layer, wherein the microelectrodes are configured to detect a signal based on a deflection in the cantilevers.

9. The cell-culture device of claim 8, further comprising one or more edge connectors coupled to the housing and electrically coupled to the one or more microelectrodes, wherein the one or more edge connectors are configured to transmit the signal to a processor.

10. The cell-culture device of claim 8, wherein the one or more microelectrodes include gold microelectrodes.

11. The cell-culture device of claim 1, wherein the layer of PE-PDMS is a 1-7% mixture of polyvinylidene fluoride-trifluoroethylene (P(VDF-TrFE)) in polydimethylsiloxane (PDMS).

12. The cell-culture device of claim 1, wherein the housing comprises a plurality of cell culture sections.

13. The cell-culture device of claim 12, wherein each of the cell culture sections comprises a plurality of compartments.

14. The cell-culture device of claim 1, wherein the housing comprises a single cell culture section having a plurality of compartments.

15. A method for measuring forces exerted by cells comprising:
seeding cells in a cell-culture device as described in claim 1;
detecting an electrical signal generated by the layer of PE-PDMS on the bottom surface of at least one of the one or more compartments, wherein the electrical signal is indicative of a mechanical movement converted to the electrical signal proportional to contractile force exerted from the cells; and
converting the electrical signal to an output of a contractile force measurement.

16. A method of making a piezoelectric sensor, comprising:
dissolving polyvinylidene fluoride-trifluoroethylene (P(VDF-TrFE)) in methyl-ethyl-ketone (MEK);
mixing polydimethylsiloxane (PDMS) and curing agent at a ratio of 10:1 into the dissolved P(VDF-TrFE) to form piezoelectric PDMS (PE-PDMS);
coating a surface of a substrate with a layer of the PE-PDMS to form the piezoelectric sensor; and
positioning a base layer including one or more microelectrodes embedded thereon beneath the layer of PE-PDMS configured to detect a signal based on a deflection in the cantilevers.

17. The method of claim 16, further comprising cutting a plurality of cantilevers in the substrate.

18. The method of claim 16, further comprising electrically coupling one or more edge connectors the one or more microelectrodes, wherein the one or more edge connectors are configured to transmit the signal to a processor.

19. The method of claim 16, wherein the one or more microelectrodes including gold microelectrodes.

20. The method of claim 16, wherein the PE-PDMS is a 1-7% mixture of P(VDF-TrFE) in PDMS.
